(19) United States
(12) Patent Application Publication
(10) Pub. No.: US 2024/0358985 A1
(43) Pub. Date: Oct. 31, 2024

(54) ADHESIVE MEDICAL PATCH PORT

(71) Applicants: BAXTER INTERNATIONAL INC., Deerfield, IL (US); BAXTER HEALTHCARE SA, Glattpark (Opfikon) (CH)

(72) Inventors: Robert Wilkie Schell, Cary, IL (US); Rajkumar Karthikeyan, Anekal Taluk (IN); Michelle Steffens, Waterford, WI (US); Hari Prasad, Bangalore (IN); Kunal Vinayak Tillu, Maharashtra (IN)

(21) Appl. No.: 18/309,239

(22) Filed: Apr. 28, 2023

(51) Int. Cl. A61M 39/02

(52) U.S. Cl. CPC A61M 39/0208

(57) ABSTRACT

Disclosed herein are systems and devices for providing injection ports to a vessel comprising a solution.

### FIELD

The present Specification relates to methods and devices for use with dialysis/intravenous administration equipment, specifically solution bags.

### BACKGROUND

Typically in Renal Peritoneal Dialysis (RPD) therapy, the “geographically fixed” injection ports/sites (FIG. 3; 330) present on the dialysis solution bags or in drain bags are not used, but are only required in cases of, for example, peritonitis, to add heparin, fibrin, or antibiotics to solution bags, or to collect the effluent in drain bags.

Thus, current devices comprise hardware that is not typically used, increasing end-user cost and unnecessarily complicating manufacture. Further, current devices and ports are expensive and fragile, and limit users to predetermined injection sites.

Therefore, improved systems, devices, and methods are desirable.

### SUMMARY

The instant disclosure provides improved systems and devices for injecting components such as drugs to solutions such as dialysis liquids. Disclosed medical patch ports replace current fixed injection sites with a simplified adhesive port that can be attached almost anywhere on a dialysis or solution vessel, such as a bag.

In disclosed embodiments, the medical patch port can be placed in a variety of locations on a dialysis liquid solution bag, thus increasing ease-of-use while replacing a costly feature of current IV or dialysis bags.

Disclosed embodiments comprise faster, simplified methods of manufacture, for example using new molding techniques.

Thus, disclosed embodiments provide increased functionality with integrity equivalent to current devices, while reducing costs and material waste.

### BRIEF DESCRIPTION OF THE FIGURES

FIG. 1 shows the current state of the art in injection site/port devices 100, with the membrane tube 110, the membrane 120, the shrink band 130, and the sleeve stopper 140.

FIG. 2 shows the current state of the art in injection site/port device production in terms of materials/sources.

FIG. 3 shows a disclosed medical patch port embodiment 310 and 320 (exploded view). The left image shows the ports 330 of current dialysis solution bags that can be replaced with disclosed embodiments.

FIG. 4 shows features of a disclosed medical patch port.

FIG. 5 shows an additional disclosed medical patch port.

FIG. 6 shows an alternate view of the medical patch port of FIG. 5.

FIG. 7 shows an additional disclosed medical patch port.

FIG. 8 shows exemplary materials for use with disclosed embodiments.

### DETAILED DESCRIPTION

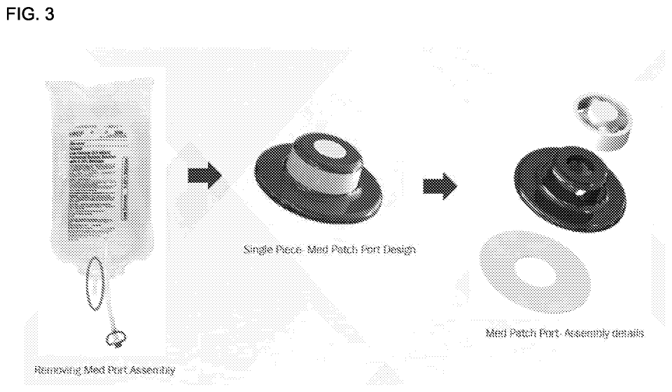

As seen in FIG. 3 (330), injection sites or ports are a key assembly in any architecture of IV or dialysis bags. They are used to provide additional medicines or drugs during therapy, which are added via injection through the injection ports of IV or dialysis bags, such that the additional drug(s) are mixed with the solution contained inside the bag.

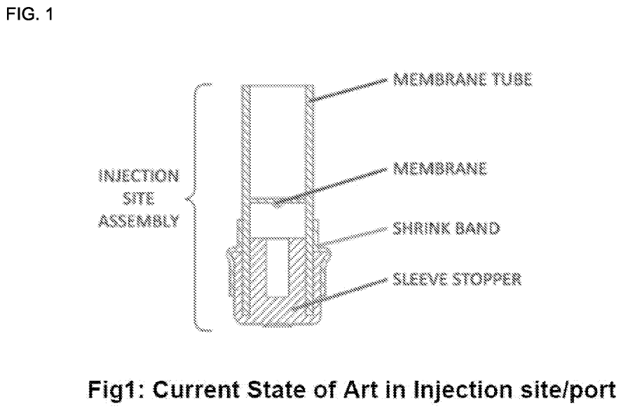

Current injection ports are made from different parts including port tubes, membranes, sleeve stoppers and a heat shrink band to hold the sleeve stopper. The sleeve stopper is the element which provides multiple re-sealing properties. These are tightly held in the port tube and heat shrink band FIG. 1 shows the injection site/port device 100, with the membrane tube 110, the membrane 120, the shrink band 130, and the sleeve stopper 140.

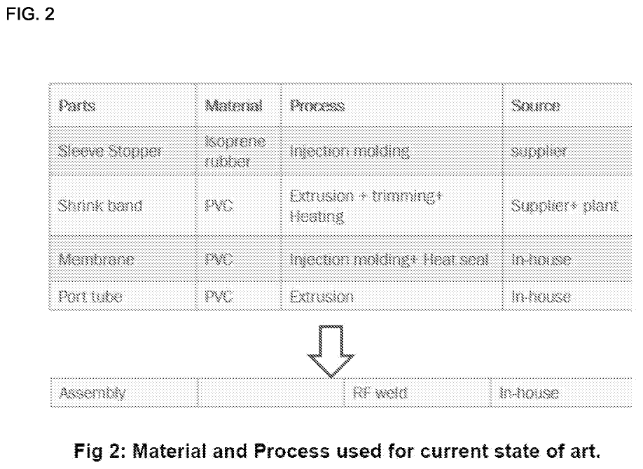

The complete injection site assembly is fixed in both the drain bag (for continuous ambulatory peritoneal dialysis [CAPD]) and in large container bag containing therapeutic solution (automated peritoneal dialysis [APD] & CAPD), are together exposed to steam sterilization (for CAPD). A typical process currently used for production is seen in FIG. 2. Further components can be assembled in the bags as well.

### Definitions

“Administration,” or “to administer” means the step of giving (i.e. administering) a device, material or agent to a subject.

“Patient” means a human or non-human subject receiving medical or veterinary care.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

### Medical Patch Ports

Disclosed embodiments comprise medical patch ports to provide injection points on solution vessels, such as IV or dialysis bags.

In embodiments, disclosed medical patch ports comprise an adhesive, wherein the user applies the medical patch port anywhere on the bag containing the solution.

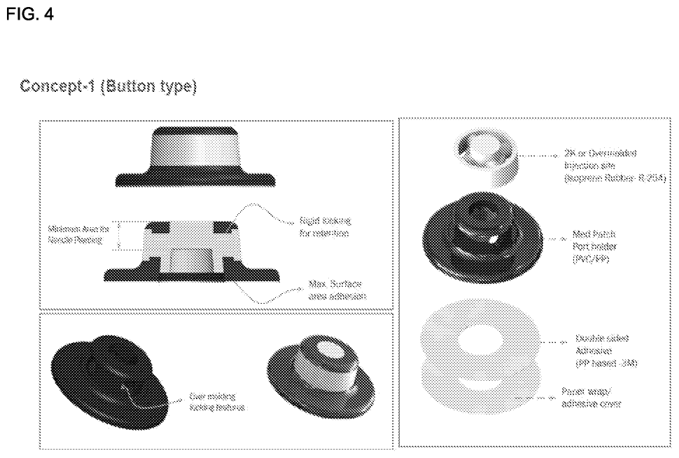

FIGS. 4-7 show medical patch port embodiments disclosed herein.

In FIG. 4, a circular medical patch port embodiment 410 comprises 4 parts. In embodiments, the medical patch port holder 420 and piercable over-molded rubber injection site component (such as an isoprene stopper 430) comprise a single part, which is 2K molded. In embodiments, means for attachment such as double-sided adhesive tape 440 is fixed on the bottom surface of the medical patch port holder and the other side is covered using a removable thin film of paper 450 or other suitable covering. The medical patch port can be applied anywhere on the solution bag that provides a regular (such as level or flat) surface.

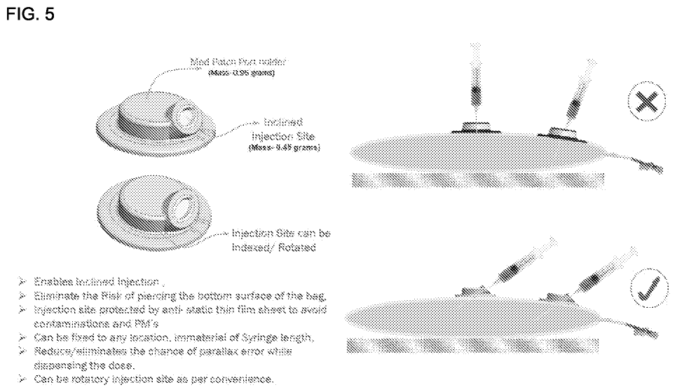

In FIG. 5, a “rotatable” embodiment 510 is disclosed. This provides the user with an angled injection option 520, so as to avoid a needle piercing of the “far” side of the bag. In embodiments, the angle between the needle's long axis during injection and the base of the medical patch port is between, for example, 75 and 35 degrees. In an embodiment, the angle between the needle's long axis during injection and the base of the medical patch port is about 45 degrees.

Disclosed embodiments comprise an integrated cap 530 which can rotate within the axis to expose the rubber injection site component. The rotatable aspect also covers the injection site component and provides a barrier or protection when the product is not in use. This can also help to avoid the parallax error caused while inserting needles. The medical patch port can be applied anywhere on the solution bag that provides a regular (such as level or flat) surface. In embodiments, the rubber injection site component further comprises an anti-static sheet.

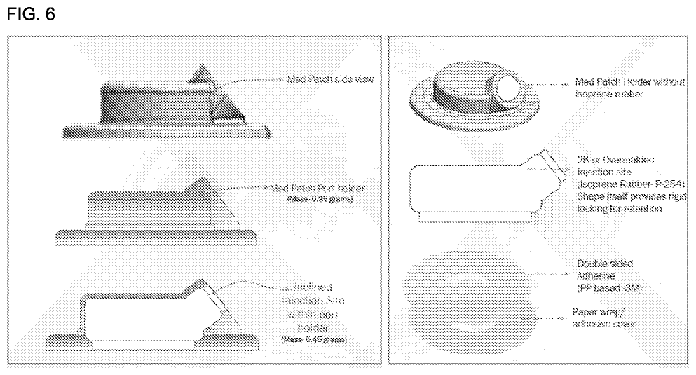

FIG. 6 shows an alternate view of the embodiment in FIG. 5. This provides the user with an angled injection option 620, so as to avoid a needle piercing of the “far” side of the bag. In embodiments, the angle between the needle's long axis during injection and the base of the medical patch port is between, for example, 75 and 35 degrees. In an embodiment, the angle between the needle's long axis during injection and the base of the medical patch port is about 45 degrees. In embodiments, means for attachment such as double-sided adhesive tape 640 is fixed on the bottom surface of the medical patch port holder and the other side is covered using a removable thin film of paper 650 or other suitable covering. The medical patch port can be applied anywhere on the solution bag that provides a regular (such as level or flat) surface.

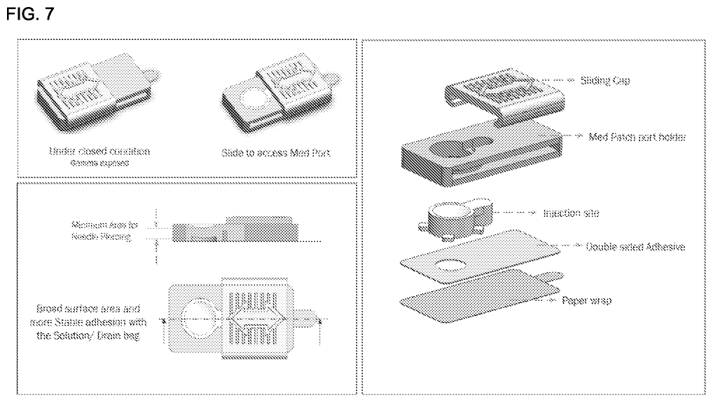

FIG. 7 shows a further embodiment comprising a sliding cap 710 which is guided along the medical patch port holder 720. In embodiments, the medical patch port holder and injection site rubber component 730 are molded together in a single molding machine at a single process, such as a 2K molding process. In embodiments, the sliding cap is then snap-fitted across the holder and guided to open and close the target area. Double-sided adhesive tape 750 is fixed on the bottom surface of the medical patch port holder and the other side is covered using a thin film paper 760. The medical patch port can be applied anywhere on the solution bag that provides a regular (such as level or flat) surface.

Another advantage provided by disclosed medical patch ports is the decrease in needle length necessary for injection; typically or PD dialysis bags, the needle must be a minimum of 30 mm in length to penetrate the inner polyvinyl chloride diaphragm in the injection port. In contrast, disclosed embodiments allow the use of shorter needles, further reducing treatment costs.

In embodiments, the entire product is exposed to a Gamma irradiation, X-ray, E-beam, steam or ethylene oxide (EO) process before or after packed in a secondary/tertiary container. In embodiments, the mechanical interlock features are provided in the medical patch port holder and the over-molded rubber is filled into the desired space, leading to an integrated structure. The counter forces for needle injection and removal are provided by these mechanical interlocking features. The materials used are common to the industry.

### Methods of Production

In embodiments, the devices are made from a single molding machine and later one side of adhesive film is attached with the base of medical patch port and the other side of the film is covered by, for example, tear away paper or other removable film.

Disclosed devices can be made from rigid resins such as PP, PVC etc., and septum are made from liquid injection molding which are over molded or 2K molded in the same machine with 2 injection units containing two different materials to process. This significantly reduces the assembly and handling cost. The mechanical holding/locking is controlled by the integrated barring features between the rigid resin and flexible resin. The proposed state of design will potentially save up to 74% of the cost involved in the current state of art.

In disclosed production methods, the method can comprise the use of multiple materials and 2K/3K or multiple-cavity hot runner molding. Disclosed embodiments can produce ports without the need for secondary operations.

In embodiments, both holder and rubber can be molded together in the same machine with twin screw barrel to process multiple materials and molded in a same tool. The high temperature holder is molded first and cooled inside the mold and then the secondary soft rubber is filled using Liquid Injection molding process. Then this integrated part is assembled with adhesive and back paper and packed into a large container (PE) which is then later exposed for Steam, Gamma, E-beam, X-ray, or etheylene oxide sterilization process.

An additional production embodiment comprises the holder being molded as a “first shot” with hard resin and then picked by robot to place it in another molder to fill the liquid silicon rubber into the second mold to achieve the desired shape. Then this integrated part is assembled with adhesive and back paper and packed into a large container (PE) which is then later exposed for Steam, Gamma, E-beam, X-ray, or etheylene oxide sterilization process.

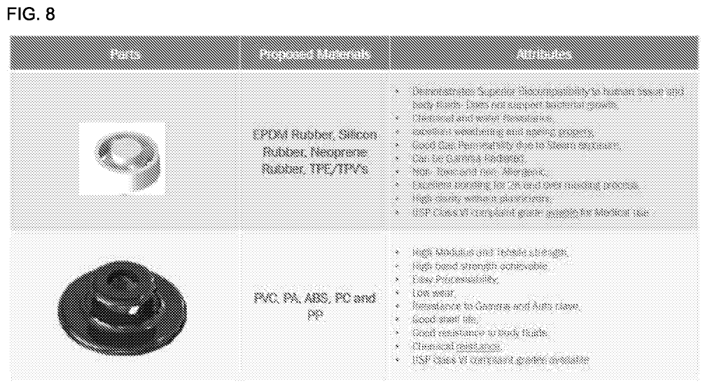

FIG. 8 shows exemplary materials for use with disclosed embodiments. However, the person of skill will understand that a number of materials used in the art would be suitable for use with disclosed embodiments.

Disclosed devices can be finished as a commercial product by the usual steps performed in the present field, for example by appropriate packaging steps. The devices can be packaged (e.g. by the addition of specific product information leaflets) into suitable containers (boxes, etc.).

### Commercial Products/Kits

According to further embodiments, the disclosed devices can also be provided in kit form combined with other components necessary for administration of the material to the patient.

The kits are designed in various forms based on the specific deficiencies they are designed to treat.

In embodiments, 25-50 medical patch ports are packaged in large anti-static bags and then exposed for Gamma, X-ray, EBeam, Etheylene oxide or Steam sterilization. These units are sold to consumers as stand alone products like disposable medical devices.

### Methods of Use

Methods of use of disclosed embodiments can comprise application to a site on a dialysis or IV solution bag where an injection is intended to be made. For example, a user can apply a disclosed medical patch port to a dialysis bag, then inject a drug or medicine into the bag via the medical patch port. The medical patch port reseals after the injection.

Disclosed medical patch ports are compatible with common materials used for dialysis and intravenous bags, including PVC, nPVC and multi-layer Polyolefin bags.

Disclosed medical patch ports are suitable for use with multi-chamber bag systems.

### EXAMPLES

The following non-limiting Examples are provided for illustrative purposes only in order to facilitate a more complete understanding of representative embodiments. This example should not be construed to limit any of the embodiments described in the present specification.

### Example 1

### Production of a Medical Patch Port

The medical patch port holder and rubber injection site component are molded together in the same machine with twin screw barrel to process multi material and mold in a same tool. The high temperature holder is molded first and cooled inside the mold and then the secondary soft rubber is filled using a Liquid Injection molding process. Then this integrated part is assembled with adhesive and back paper and packed into a large container (PE) which is then later exposed for Steam, Gamma, E-beam, X-ray, or Etheylene oxide sterilization process.

### Example 2

### Production of a Medical Patch Port

The medical patch port holder is molded in a first mold with hard resin and then picked by a robot to place it in the another molder to fill the liquid silicon rubber injection site component into the second mold to achieve the desired shape. Then this integrated part is assembled with adhesive and back paper and packed into a large container (PE) which is then later exposed for Steam, Gamma, E-beam, X-ray, or Etheylene oxide sterilization process.

### Example 3

### Use of a Medical Patch Port

A disclosed medical patch port is applied to a desired injection location site on a dialysis bag. The adhesive is exposed by removing the paper layer, and the port is attached to the dialysis bag. Following attachment of the medical patch port, material is injected into the bag through the medical patch port.

### Example 3

### Use of a Medical Patch Port

A disclosed medical patch port is applied to a desired injection location site on a bag with an intravenous solution. The adhesive is exposed by removing the paper layer, and the port is attached to the bag. Following attachment of the medical patch port, material is injected into the bag through the medical patch port.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A medical patch port for providing injection access to a flexible vessel comprising a solution, said medical patch port comprising an adhesive.

2. The medical patch port of claim 1, wherein said medical patch port comprises a medical patch port holder.

3. The medical patch port of claim 2, wherein said medical patch port comprises a piercable injection site component.

4. The medical patch port of claim 3, wherein said medical patch port comprises a sliding component to provide access to the injection site.

5. The medical patch port of claim 3, wherein said medical patch port comprises a rotatable medical patch port holder.

6. A method for providing a re-sealable injection site on a vessel comprising a solution, said method comprising applying to the intended injection site a medical patch port comprising an adhesive.

7. The method of claim 6, wherein said medical patch port comprises a medical patch port holder.

8. The method of claim 7, wherein said medical patch port comprises a piercable injection site component.

9. The method of claim 8, wherein said medical patch port comprises a sliding component to provide access to the injection site.

10. The method of claim 8, wherein said medical patch port comprises a rotatable medical patch port holder.

11. A kit for use in providing a re-sealable injection site on a vessel comprising a solution, said kit comprising a medical patch port comprising an adhesive.

12. The kit of claim 11, wherein said medical patch port comprises a medical patch port holder.

13. The kit of claim 12, wherein said medical patch port comprises a piercable injection site component.

14. The kit of claim 13, wherein said medical patch port comprises a sliding component to provide access to the injection site.

15. The kit of claim 13, wherein said medical patch port comprises a rotatable medical patch port holder.
